(19) United States
(12) Patent Application Publication
(10) Pub. No.: US 2005/0129756 A1
(43) Pub. Date: Jun. 16, 2005

(54) UV-stable, liquid or semisolid transdermal pharmaceutical preparation with light sensitive active ingredient

(76) Inventors: Hans-Peter Podhaisky, Jena (DE); Stefan Bracht, Jena (DE)

Correspondence Address: STRIKE, STRIKER & STENBY, 103 EAST NECK ROAD, HUNTINGTON, NY, 11743, US

(21) Appl. No.: 11/006,434

(22) Filed: Dec. 7, 2004

Related U.S. Application Data
(60) Provisional application No. 60/533,277, filed on Dec. 30, 2003.

(30) Foreign Application Priority Data
Dec. 10, 2003 (FR) 03 028 353.3

(51) Int. Cl. A61K 31/56; A61K 9/20

(52) U.S. Cl. 424/464; 514/171

(57) ABSTRACT

The semisolid or liquid pharmaceutical preparation for transdermal administration contains at least one UV-light sensitive pharmaceutically active ingredient and at least one UV-absorbing substance, which is present in an amount that does not have pharmacological activity and in dissolved or dispersed form. This semisolid of liquid pharmaceutical preparation is preferably a hydroalcoholic gel, whose gel base includes water, alcohol, at least one gel-forming polymer and additional ingredients as needed. This pharmaceutical preparation is prepared with a UV-light sensitive pharmaceutically active ingredient, so that the application system has a high stability without the disadvantages of the known semisolid transdermal application forms. The injurious side effects, such as absorption of the UV-light protecting ingredient in the body, resulting from the intended light protection are reduced as much as possible.

### CROSS-REFERENCE

U.S. Provisional Application No. 60/533,277, filed Dec. 30, 2004, discloses inventive subject matter, which is also disclosed herein.

### BACKGROUND OF THE INVENTION

The subject matter of the present invention is a semisolid or liquid pharmaceutical preparation for transdermal administration comprising a pharmaceutically active ingredient that is sensitive to UV radiation and at least one UV-absorbing auxiliary substance (UV-absorber).

The semisolid and liquid transdermal pharmaceutical preparations according to the invention especially include transdermal gels, whose gel base comprises water, alcohol, at least one gel forming polymer and other ingredients as needed. Those gels are called hydrogels. The UV-absorbing substance according to the invention is present in the gel in dissolved or dispersed form.

From the patent and professional literature it is known that transdermal administration of pharmaceutically active ingredients has various advantages in comparison to oral administration of a medicine, such as
no first pass metabolizing of the active ingredient due to passage through the gastrointestinal tract;
releasing active ingredient uniformly over several hours, according to the depot operation of the skin; and
keeping plasma levels of the active ingredient uniform because of the uniform release.

Currently known transdermal therapeutic systems can be formulated in two different forms for administration. There are large numbers of therapeutic patches, which contain the active ingredient in dissolved or suspended form in a matrix.

In recent years semisolid transdermal pharmaceutical preparations and, above all, gels have become established in this field. In contrast to the transdermal patch this non-occlusive form has the advantage that skin compatibility is improved. Other advantages are individual dosability and that the user is not viewed as sick or is not stigmatized as a person needing treatment.

Various pharmaceutically active ingredients, which are suitable for transdermal application, (e.g. nifedipine, nicotine, arylpropionic acid and benzophenone derivatives, gestoden, levonorgestrel, estradiol and other hormones suitable for transderamal application) are light sensitive. Light sensitive substances absorb radiation within wavelength ranges in the spectrum corresponding to visible light, and, above all, in the ultraviolet range (UV, 280 to 400 nm). Photochemical decomposition of the active ingredient and in extreme cases loss of effectiveness of the active ingredient can be caused by exposing pharmaceutical preparations with a light sensitive active ingredient to light. Furthermore the decomposition products can cause phototoxic or photoallergic reactions in the skin. For example, it is known that ketoprofen gels like other aryl propionic acid and benzophenone derivatives lead to frequent occurrence of phototoxic and partially photoallergic skin reactions, which are ascribed to the photosensitization properties of these classes of substances. Suitable protective steps must be taken for transdermal preparations with light sensitive active ingredients in order to prevent these types of decomposition processes.

The possibility exists in the field of transdermal plasters to spatially separate the UV-absorbing substance from the active ingredient based on layered structures typically with at least one effective ingredient-containing adhesive layer and a rear side plastic foil (backing layer). Solutions according to the invention for this situation are disclosed in EP 1449526. There is a possibility, especially with plasters with a layer structure to provide the UV-absorbing substance in a layer, which is above the effective ingredient layer, in the sense that the damaging light rays must first penetrate the UV-absorbing layer before they reach the effective ingredient-containing layer. Thus the effective ingredient is especially well protected by the UV-absorbing substance. In other transdermal pharmaceutical preparations, such as solutions and semisolid preparations, this type of protection cannot be used, since in these other preparations there is no possibility for definite spatial separation of the effective ingredient from the UV-absorbing substance.

Currently light protection is provided in the currently known transdermal gels only to the extent that the preparations are packaged in light impermeable packages, chiefly aluminized edge-sealing sacs or sachets. These methods of protecting semisolid pharmaceutical preparations (TTS) for transdermal administration against light are effective only during storage, and are generally insufficient. They assume that the light sensitive active ingredient is no longer exposed to sunlight after its application to the skin. Protection from sunlight can occur e.g. by wearing clothing over the application area on the skin. However this puts an undesirable limitation on the user, especially in regions with a warm climate or during warm periods of the year when less clothing is worn.

It is known that the UV portion of sunlight penetrates the skin. Thus UVB light (280-320 nm) penetrates the entire epidermis and reaches the Stratum basal. The longer wavelength UVA light (320-400 nm) penetrates to the connective tissue or collagen-containing tissue. Transdermal gels are generally applied over a comparative large area (100 to 200 cm² and more). The complete active ingredient transport from the outermost layer of the skin (Stratum corneum) to the systematic circulation takes up to 24 hours. Above all the Stratum corneum functions as skin penetration barrier and as effective ingredient reservoir, which stores comparatively large portions of the drug for several hours. The entire UV spectrum penetrates this region and causes photochemical decomposition reactions.

From this standpoint the light sensitivity problems encountered by transdermal gels are greater than those of transdermal patches during administration. The exposed application surfaces are generally much smaller with transdermal patches and they are provided with a least one outer backing layer made of plastic foil, which generally always provides minimal UV protection.

Semisolid transdermal systems (gels) are known from the patent literature with pharmaceutical, also light sensitive, active ingredients.

WO-A1-03/082960 discloses the manufacture of a gel, which can contain a pharmaceutically active ingredient. A gel containing diclofenac is described in WO-A1-01/60399. WO-A2-02/051421 describes a gel composition with at least one androgenic steroid for treatment and/or prophylaxis of hypogonadism. However the light sensitivity problems of the active ingredients or measures to protect the active ingredients are not covered or considered in any of these patent documents. All these patent documents are not related to the present problems regarding light sensitivity and light protection of the semisolid transdermal pharmaceutical preparation.

### SUMMARY OF THE INVENTION

It is an object of the present invention to provide a semisolid or liquid pharmaceutical preparation for transdermal administration containing a light sensitive pharmaceutically active ingredient, which has a great stability and does not suffer from the disadvantages of conventional semisolid pharmaceutical preparations for transdermal administration.

It is also an object of the present invention to provide a semisolid or liquid pharmaceutical preparation for transdermal administration containing a light sensitive pharmaceutically active ingredient, which keeps injurious side effects, e.g. from absorption of light protective agents in the body, resulting from the intended light protection as small as possible.

According to the invention these objects are attained by a semisolid or liquid pharmaceutical preparation for transdermal administration comprising at least one UV-light sensitive pharmaceutically active ingredient and at least one UV-absorbing substance, which is present in dissolved or dispersed form and which is used only in concentrations such that it has no pharmacological activity, but has sufficient UV protective action.

According to preferred embodiments of the invention the semisolid or liquid pharmaceutical preparation for transdermal administration can be a hydroalcoholic gel. In other embodiments other pharmaceutical preparations, such as creams, solutions and suspensions, have also proven advantageous.

Surprisingly a spatial separation between the pharmaceutically active ingredient or ingredients and the UV-absorbing substance can be achieved by suitable selection of the UV-absorbing substance under the following administration conditions.

While the transdermal active ingredient necessarily belongs to the group of skin permeable pharmaceutically active ingredients, the UV-absorbing substance according to the invention may be a UV-absorbing substance, which penetrates or permeates the skin as little as possible. Since the active ingredient and the absorbing substance are initially mixed with each other in the transdermal gel or solution in the pharmaceutical preparation according to the invention, during subsequent diffusion in and through the skin a spatial separation of these ingredients occurs. The more poorly penetrating UV-absorbing substance remains back in the outer skin layers and rests between the deeper active ingredient and the UV radiation acting on the outside of the body.

The logarithm of the distribution coefficient between 1-octanol and water (Log P_{Oct/water} in the following designated by Log P) and the molecular weight, have a special value in decisions regarding selection of a suitable UV-absorbing substance to protect the semisolid or liquid pharmaceutical preparations for transdermal administration.

Furthermore in the semisolid or liquid transdermal pharmaceutical preparations according to the invention the distribution coefficient or coefficients (Log P) of the UV-absorbing substance or absorbing substances can be greater than 3.0 or less than 1.0, preferably greater than 5.0 or less than 0.0.

UV-absorbing substances are preferred, which can penetrate or permeate the skin only to a small extent, since they are either too lipophilic or too hydrophilic.

In preferred embodiments the molar mass of the UV-absorbing substance or absorbing substances in the semisolid transdermal pharmaceutical preparations should be greater than 250 g/mol, preferably greater than 500 g/mol. UV-absorbing substances with a molecular weight above 500 g/mol only have a reduced penetration and permeation for skin.

In preferred embodiments the semisolid or liquid transdermal pharmaceutical preparations can contain from 1 to 10 percent by weight of the UV-absorbing substance or absorbing substances, preferably from 2 to 5 percent.

In so far as the semisolid transdermal pharmaceutical preparations according to the invention contain volatile components, such as ethanol, methanol, propanol or DSMO, the percentage amount ranges relate to the sum of the nonvolatile vehicle ingredients or, in other words, to the amount of the formulation remaining on the skin after all volatile ingredients leave it.

A UV-absorbing substance or substances can be found, whose absorption maximum or maxima are within a wavelength range, which is responsible for photochemical decomposition of the active ingredient to be protected. In as much as protection is required over a wide UV spectral range and/or for absorption maxima of the effective ingredient within that range, it has proven advantageous to combine two UV-absorbing substances with different absorption maxima.

Furthermore the substances absorbing in the UV range included in the semisolid transdermal pharmaceutical preparations can be selected from the group consisting of p-aminobenzoic acid and aminobenzoic acid derivatives, preferably 4-dimethylaminobenzoic acid-2-ethyl-hexylester and cinnamic acid and its derivatives, preferably 4-methoxycinnamic acid isoamyl ester and 3-benzyliden-boranan-2-one and benzyliden-bornan-2-one derivatives, preferably 3-(4′)-methylbenzyliden-bornan-2-one, 3-(4-sulfo)benzylidenebornan-2-one, and salicylic acid derivatives, preferably 4-isopropylbenzylsalicylate, salicylic acid-2-ethylhexylester, 3,3,5-trimethylcyclohexylsalicylate, and 3-imidazole-4-yl-acrylic acid and its esters, 2-phenylenebenzimidazole-5-sulfonic acid, methylene bis-benzotriazolyl-tetramethylbutylphenol, 2-cyano-3,3-diphenylacrylic acid, butyl-methoxydibenzoyl methane and benzophenone or benzophenone derivatives, preferably benzophenone-3, benzophenone-4.

The following UV-absorbing substances, which have a center or gravity in the UVA range, stand out as especially suitable in the present invention: 4-bis-(polyethoxy)-aminobenzoic acid polyethoxyethyl ester, 2,2′-dihydroxy-4,4′-dimethoxybenzophenone-5,5′-disodium sulfonate, 4-methoxycinnamic acid-2-ethylhexylester, 3-(4′-trimethylammonium)-benzyliden-bornan-2-one methyl sulfate, 2, 4, 6-trianiline-p-(carbo-2′-ethyl-hexyl-1′-oxy)-1, 3, 5-triazine, dioctyl butamidotriazone, bis-ethylhexyloxyphenol methoxy-phenyltriazone, terephthaloylidene dicamphor sulfonic acid, polymers of N-[2-(2-oxoborn-3-ylidenmethyl)benzyl]acrylamide and N-[4-(2-oxoborn-3-ylidene-methyl)-benzyl]-acrylamide, drometriazol trisiloxane and 2,2′-(1,4-phenylen)-bis-(1H-benzimidazol-4,6-disulfonic acid, monosodium salt).

The trademarks for specially preferred UV-absorbing substances are TINUVIN® 326, TINOSORB® S and MEXORYL® TL.

In the case of UV protective substances, which react as acids, such as carbonic acids or sulfonic acids, their pharmaceutically acceptable salts, such as preferably and without claiming completeness, K-, Na- and triethanolamine (TEA) salts, can be used.

Furthermore the semisolid transdermal pharmaceutical preparations according to the invention can contain at least one hormone as pharmaceutically effective active ingredient.

Further the pharmaceutically active ingredient in the semisolid transdermal pharmaceutical preparations according to the invention can be a gestagen or gestagens, preferably gestoden and/or levonorgestrel, or an estrogen or estrogens, preferably ethinyl estradiol, or a combination of estrogen and gestagen, preferably gestoden and ethinyl estradiol, and also an androgen or androgens, preferably testosterone, methyltestosterone, methyl-nortestosterone (MENT) or 7α-methyl-11β-fluoro-19-nortestosterone (ef-MENT).

Also the semisolid transdermal pharmaceutical preparations according to the invention can be transparent and colorless in preferred embodiments.

The gel formers of the semisolid transdermal pharmaceutical preparations preferably include celluloses, preferably hydroxyethyl cellulose, hydroxypropyl cellulose, methyl cellulose, methylhydroxyethyl cellulose, methylhydroxypropyl cellulose, Na-carboxymethylcellulose; chitosan-EDTA, highly dispersed silicon dioxide, precipitated silicon dioxide, bentonite; starch, preferably corn starch, rice starch, potato starch, wheat starch; carboxymethylamylopectin-sodium, traganth, alginate; polyacrylates, polymethacrylate, polyacrylate-polyalkylacrylate cross polymer, acryloyldimethyl taurate/vinyl pyrrolidone copolymer, polyvinyl alcohol or polyvinyl pyrrolidone.

The invention will be explained further with the help of the following examples, which should not be considered to limit the claims appended hereinbelow.

### EXAMPLES

### Example 1

Ketoprofen is a non-steroidal anti-inflammatory (NSAID), which is used for treatment of swelling and inflammation of the soft regions in the vicinity of the joints (e.g. tendons, sinews, connective tissue and joint caps), especially near the shoulder and elbow, which occur, because of sport and accident injuries, such as bumps or collisions, sprains, tears and pulls. Phototoxic and photoallergic skin reactions under the influence of sunlight are known to occur with this sort of gel.

The effective ingredient, ketoprofen, is protected from light-induced decomposition by the use of the UV-absorbing substance, Uvinul DS 49. Uvinul DS 49 is 2,2′-dihydroxy-4,4′-dimethoxy-benzophenone-5,5′-disodium sulfonate. The semisolid therapeutic transdermal system had the following composition.

| | |
|---|---|
| Ketoprofen | 2.5% |
| Carbopol 940 | 1% |
| Uvinul DS 49 | 2% |
| Triethanolamine (TEA) | 1% |
| Isopropanol | 20% |
| Propylene glycol | 20% |
| EDTA | 0.1% |
| Water | To 100% |

Uvinl DS 49 has a molecular weight of about 478.4 g/mol and a calculated Log P value of −1.9.

The gel base was made from carbopol, TEA and water according to the generally accepted pharmaceutical recipe. After that the Uvinul DS 49 and EDTA were worked into this gel base. Isopropanol, propylene glycol and ketoprofen were measured out and subsequently worked into the gel base.

### Example 2

Gestoden-containing preparations can be formulated for hormone replacement therapy. The active ingredient can decompose under UV-light and thus there is an inherent loss in the amount of active ingredient. The preparation contains Uvinul MC 80 as UV protective agent. UV MC 80 is 4-methoxycinnamic acid 2-ethylhexyl ester. The semisolid transderamal pharmaceutical preparation according to the invention containing these ingredients had the following composition.

| | |
|---|---|
| Gestoden | 1% |
| Uvinul MC 80 | 2% |
| Diethylene glycol monoethyl ether | 10% |
| Isopropyl myristate | 10% |
| Ethanol | 70% |
| Hydroxypropyl cellulose | 1.5% |
| Water | To 100% |

Uvinl MC 80 has a molecular weight of about 290 g/mol and a calculated Log P value of 5.37.

Gestoden and Uvinul MC 80 were dissolved in ethanol. Subsequently diethyleneglycol monoethyl ether, isopropyl myristate and water were added and mixed well. After that hydroxypropyl cellulose was worked portion-wise into the mixture to form the transdermal gel. Then the resulting pharmaceutical preparation was allowed to swell sufficiently.

### Example 3

Ethinyl estradiol-containing preparations can be formulated for hormone replacement therapy. The active ingredient can decompose under UV-light and thus there is an inherent loss in the amount of active ingredient. The preparation contains Tinosorb S as UV protective agent. Tinosorb S is bis-ethylhexyl-oxyphenyl methoxyphenyltriazine. The semisolid pharmaceutical preparation for transdermal administration according to the invention containing these ingredients has the following composition.

| | |
|---|---|
| Ethinyl estradiol | 0.2% |
| Tinosorb S | 2% |
| Diethylene glycol monoethyl ether | 10% |
| Isopropyl myristate | 10% |
| Ethanol | 70% |
| Hydroxypropyl cellulose | 1.5% |
| Water | To 100% |

Tinosorb S has a molecular weight of about 627.80 g/mol and a calculated Log P value of 9.

Ethinyl estradiol and Tinosorb S were dissolved in ethanol. Subsequently diethyleneglycol monoethyl ether, isopropyl myristate and water were added and mixed well. After that hydroxypropyl cellulose was worked portion-wise into the mixture to form the transdermal gel. Then the resulting pharmaceutical preparation was allowed to swell sufficiently.

### Example 4

A combination of the hormones, ethinyl estradiol and gestoden, can be used for contraception. Decomposition of these effective ingredients occurs under UV light and thus effective pharmaceutical action is reduced or lost.

Ethinyl estradiol and gestoden-containing preparations can be formulated with UV-light protection according to the invention. This example of a preparation according to the invention also contains Tinosorb S as UV-protective agent. Tinosorb S is bis-ethylhexyl-oxyphenyl methoxyphenyltriazine. The semisolid pharmaceutical preparation for transdermal administration according to the invention containing these ingredients had the following composition.

| | |
|---|---|
| Ethinyl estradiol | 0.4% |
| Gestoden | 1% |
| Tinosorb S | 2% |
| Diethylene glycol monoethyl ether | 10% |
| Propylene glycol | 5% |
| Carbopol 940 | 1% |
| Ethanol | 50% |
| Triethanolamine (TEA) | 1% |

Ethinyl estradiol, gestoden and Tinosorb S were dissolved in the ethanolic phase. The gel base was made from carbopol, TEA and water according to the generally accepted pharmaceutical recipe. Propylene glycol and diethylene glycol monoethyl ether were measured out and subsequently the ethanolic phase was worked into the gel.

### Example 5

The effective ingredient 7α-methyl-11β-fluoro-19-nortestosterone (ef-MENT) can be used for hormone replacement therapy in hypogonadal men. Ef-MENT-containing preparations can be formulated with UV-light protection according to the invention. This example according to the invention thus contains Tinosorb S as UV protective agent. The semisolid transderamal pharmaceutical preparation according to the invention containing these ingredients had the following composition.

| | |
|---|---|
| Propylene carbonate | 10.0% |
| Ethanol | 54.0% |
| Pure water | ˜29.0% |
| Ef-MENT | 0.8% |
| Tinosorb S | 1.0% |
| Acrylate/C 10-30 alkylacrylate | 0.8% |
| Crosspolymer (Permulen TR-1) | |
| Methylcellulose (Tylopur MH 1000) | 0.5% |
| Glycerol 86% | 1.0% |
| Cyclomethicone | 1.5% |
| Isopropyl myristate | 0.5% |
| Tris-(hydroxymethyl)aminomethane to pH = 5.8 | |

EF-MENT and Tinosorb S were first dissolved in ethanol 96%. Permulen TR-1 and HPC (Klucel HF) were swollen in this ethanolic solution. Subsequently propylene carbonate was added and mixed by stirring. Then isopropyl myristate was added and the mixture was mixed again. The entire mixture was supplied to a mixture/homogenizer system by means of a funnel and briefly stirred. After that the mixture was homogenized (e.g. with a rotor-stator homogenizer) for 1 minute at 2000 to 3500 rpm. Glycerol 86% and the entire quantity of pure water were added as a schlieren-free mixture in several partial steps (soaked in) and the gel clearly swelled further. It was stirred (about 5 minutes) until a clear clarification of the gel was observed. Subsequently the cyclomethicone was worked in with stirring into the mixture. Subsequently a 10% aqueous tromethamine solution was added for neutralization with stirring in three partial steps. The product was homogenized still more for 2 minutes at 2700 rpm after this working in was finished.

Unless otherwise indicated, all percentages are percentages by weight.

The disclosures in European Patent Application 03 028 353.5, filed Dec. 10, 2003, are incorporated here by reference. This European Patent Application describes the invention described hereinabove and claimed in the claims appended hereinbelow and provides the basis for a claim of priority for the instant invention.

While the invention has been illustrated and described as embodied in a UV-stable liquid or semisolid transdermal pharmaceutical preparation with light-sensitive active ingredient, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

1. A semisolid or liquid pharmaceutical preparation for transdermal administration, said preparation comprising at least one UV-light sensitive pharmaceutically active ingredient and at least one UV-absorbing substance, wherein the at least one UV-absorbing substance is present only in an amount such that the at least one UV-absorbing substance does not have pharmacological activity and is present in dissolved or dispersed form.

2. The semisolid or liquid pharmaceutical preparation as defined in claim 1, consisting of a hydroalcoholic gel.

3. The semisolid or liquid pharmaceutical preparation as defined in claim 1, wherein said at least one UV-absorbing substance has a distribution coefficient (Log P) either greater than 3.0 or less than 1.0.

4. The semisolid or liquid pharmaceutical preparation as defined in claim 3, wherein said distribution coefficient is greater than 5.0 or less than 0.0.

5. The semisolid or liquid pharmaceutical preparation as defined in claim 1, wherein said at least one UV-absorbing substance has a molecular mass greater than 250 g/mol.

6. The semisolid or liquid pharmaceutical preparation as defined in claim 5, wherein said molecular mass is greater than 500 g/mol.

7. The semisolid or liquid pharmaceutical preparation as defined in claim 1, wherein said at least one UV-absorbing substance is present in an amount of from 1 to 10% by weight, based on a total amount of non-volatile ingredients present.

8. The semisolid or liquid pharmaceutical preparation as defined in claim 7, wherein said amount of said at least one UV-absorbing substance is from 2 to 5% by weight, based on said total amount of said non-volatile ingredients present.

9. The semisolid or liquid pharmaceutical preparation as defined in claim 1, wherein said at least one UV-absorbing substance, which absorbs in a UV spectral range, is p-aminobenzoic acid, 4-dimethylaminobenzoic acid-2-ethyl-hexyl ester, 4-bis-(polyethoxy)-aminobenzoic acid polyethoxyethyl ester, cinnamic acid, 4-methoxycinnamic acid isoamyl ester, 4-methoxycinnamic acid-2-ethylhexylester, 3-benzylidenebornan-2-one, 3-(4′)-methylbenzyliden-bornan-2-one, 3-(4-sulfo)-benzylidenebornan-2-one, 3-(4′-trimethylammonium)-benzylidenbornan-2-one methyl sulfate, polymers of N-[2-(2-oxoborn-3-ylidenmethyl)benzyl]acrylamide, polymers of N-[4-(2-oxoborn-3-ylidene-methyl)benzyl]acrylamide, 4-isopropyl-benzylsalicylate, salicylic acid-2-ethylhexylester and/or 3,3,5-trimethylcyclohexyl-salicylate.

10. The semisolid or liquid pharmaceutical preparation as defined in claim 1, wherein said at least one UV-absorbing substance, which absorbs in a UV spectral range, is 2,4,6-trianiline-p-(carbo-2′-ethyl-hexyl-1′-oxy)-1,3,5-triazine, dioctyl butamidotriazine, bis-ethylhexyl-oxyphenol methoxyphenyltriazone, 3-imidazol-4-yl-acrylic acid, esters of 3-imidazol-4-yl-acrylic acid, 2-phenylen-benzimidazol-5-sulfonic acid, 2,2′-(1,4-phenylen)-bis-(1H-benzimidazol-4,6-disulfonic acid, monosodium salt), methylene bis-benzotriazolyl-tetramethyl-butylphenol, drometriazole trisiloxane, 2-cyano-3,3-diphenylacrylic acid, terephthaloylidene dicamphor sulfonic acid, butylmethoxydibenzoyl-methane, benzophenone, benzophenone-3, benzophenone-4 and/or 2,2′-dihydroxy-4,4′-dimethoxy-benzophenon-5,5′-disodium sulfonate.

11. The semisolid or liquid pharmaceutical preparation as defined in claim 1, wherein said at least one UV-light sensitive pharmaceutically active ingredient is at least one hormone.

12. The semisolid or liquid pharmaceutical preparation as defined in claim 1, wherein said at least one UV-light sensitive pharmaceutically active ingredient is a gestagen.

13. The semisolid or liquid pharmaceutical preparation as defined in claim 12, wherein said gestagen is gestoden or levonorgestrel.

14. The semisolid or liquid pharmaceutical preparation as defined in claim 1, wherein said at least one UV-light sensitive pharmaceutically active ingredient is an estrogen.

15. The semisolid or liquid pharmaceutical preparation as defined in claim 14, wherein said estrogen is ethinyl estradiol.

16. The semisolid or liquid pharmaceutical preparation as defined in claim 1, wherein said at least one UV-light sensitive pharmaceutically active ingredient comprises a combination of gestagen and estrogen.

17. The semisolid or liquid pharmaceutical preparation as defined in claim 1, wherein said combination of said gestagen and said estrogen is a combination of gestoden and ethinyl estradiol.

18. The semisolid or liquid pharmaceutical preparation as defined in claim 1, wherein said at least one UV-light sensitive pharmaceutically active ingredient is an androgen.

19. The semisolid or liquid pharmaceutical preparation as defined in claim 18, wherein said androgen is testosterone, methyltestosterone, methyl-nor-testosterone (MENT) or 7α-methyl-11 β-fluoro-19-nortestosterone (ef-MENT).

20. The semisolid or liquid pharmaceutical preparation as defined in claim 1, in the form of a colorless and transparent preparation.

21. The semisolid or liquid pharmaceutical preparation as defined in claim 20, wherein said preparation is a gel.

22. The semisolid or liquid pharmaceutical preparation as defined in claim 1, 20 or 21, further comprising a gel former and wherein said gel former is hydroxyethyl cellulose, hydroxypropyl cellulose, methyl cellulose, methylhydroxyethyl cellulose, methylhydroxypropyl cellulose, Na-carboxymethylcellulose, chitosan-EDTA, highly dispersed silicon dioxide, precipitated silicon dioxide, bentonite, corn starch, rice starch, potato starch, wheat starch, carboxymethylamylopectin-sodium, tragacanth, alginate, polyacrylate, polymethacrylate, polyacrylate-polyalkylacrylate cross polymer, acryloyldimethyl taurate/vinyl pyrrolidone copolymer, polyvinyl alcohol or polyvinyl pyrrolidone.

23. The semisolid or liquid pharmaceutical preparation as defined in claim 1, wherein said at least one UV-light sensitive pharmaceutically active ingredient penetrates or permeates human skin to a much greater extent and depth than said at least one said UV-absorbing substance during transdermal administration, so that said at least one UV-absorbing substance is contained in skin layers substantially above said at least one UV-light sensitive pharmaceutically active ingredient absorbed in said human skin, in order to at least reduce an amount of UV radiation reaching said at least one UV-light sensitive pharmaceutically active ingredient.

24. The semisolid or liquid pharmaceutical preparation as defined in claim 23, wherein said at least one UV-absorbing substance has a distribution coefficient (Log P) either greater than 3.0 or less than 1.0 and said at least one UV-absorbing substance has a molecular mass greater than 250 g/mol.

25. The semisolid or liquid pharmaceutical preparation as defined in claim 23, wherein said at least one UV-light sensitive pharmaceutically active ingredient is a steroid hormone.
